(19) United States
(12) Patent Application Publication
(10) Pub. No.: US 2017/0216532 A1
(43) Pub. Date: Aug. 3, 2017

(54) INJECTING DEVICE WITH DOSE RESETTING MECHANISM

(71) Applicant: COPERNICUS SP. Z O.O., Szczecin (PL)

(72) Inventor: Adam STEFANSKI, Gniezno (PL)

(21) Appl. No.: 15/489,697

(22) Filed: Apr. 17, 2017

Related U.S. Application Data
(63) Continuation of application No. 14/377,479, filed on Aug. 7, 2014, now abandoned.

(30) Foreign Application Priority Data
Feb. 8, 2012 (PL) PL398051

(51) Int. Cl. A61M 5/315; A61M 5/20

(52) U.S. Cl. CPC A61M 5/31543; A61M 5/31511; A61M 5/31553; A61M 5/20; A61M 5/31583; A61M 2005/2026

(57) ABSTRACT

Injecting device with dose resetting mechanism enables release energy accumulated in spring means (17) and causes automatically the back movement of an indicating element (16) to its initial position without causing any axial movement of a threaded piston rod (10), said actions being initiated by one movement of a dose setting element (4) in the direction opposite to that when setting a dose. Reset clutch (13) is made as an axially sliding, shaped coupling element moved by means of the dose setting element (4), and moreover this reset clutch (13) is functionally connected with a drive unit destined to co-operate with the spring means (17) in such a way that when this reset clutch (13) is released, said drive unit is disconnected from the spring means (17) retaining the connection of this spring means (17) with the indicating element (16).

This invention concerns an injecting device having a dose resetting mechanism, preferably for administration of insulin. The device is applicable for multiple administration of set portions of medicine from an exchangeable container, with the possibility of resetting the last set dose and setting another one.

From WO 2010089417 an injecting device is known designed for multiple administration of set portions of medicine from an exchangeable container. A dose setting element cooperates in this device by means of a ratchet clutch with a driving device. In particular, this device comprises a control sleeve connected with a cylindrical indicating element, a knob, a clutch sleeve, a torsion spring, a spring blockade, a threaded piston rod, a ratchet, a nut, a threaded piston rod blockade, a trigger, and a housing for the whole mechanism. The known solution does not reveal a mechanism for total resetting of a set dose.

From WO 9307922 an injecting device is known comprising a one-way clutch between a dose setting mechanism and a piston driving means, said clutch blocking any possibility of changing the rotary motion when setting a dose into a shift motion of a piston driving element while setting a dose. In order to reset the formerly set dose, said one-way clutch disconnects itself by its axial motion created by means of a rising fork. However, in order to reset the dose, an unwieldy and complex operation for the user has to be performed which comprises simultaneous activating a resetting mechanism in one part of the device together with manually pressing a knob situated at the end of the whole device.

In EP1351732 an automatic injecting device is described with a resetting mechanism, comprising a housing, a driving unit, a spring means, a dose setting unit placed in the housing as well as a trigger displaceable in relation to the housing. In this solution there is a possibility to selectively correct a dose by relocating a dose setting element in the opposite direction to the dose setting direction. When the whole dose has to be reset, the dose setting element made as a knob is repeatedly turned manually until the zero position has been achieved.

In EP1954337 an automatic injecting device is described comprising a dose setting element made as a knob rotatably coupled on a housing as well as a trigger placed slidably in relation to the housing and intended to release the energy accumulated in a spring in order to put a nut driving a threaded piston rod into rotation. This device has also a mechanism releasing energy accumulated in said spring without causing the axial motion of the threaded piston rod, said mechanism being made as an additional button connected with said housing, said button being axially movable in relation to the housing. A dose can be corrected in this solution by pressing the said button, causing simultaneously the turning of the knob in the direction opposite to the dose setting direction. When the whole dose has to be reset, the above action should be repeated until the zero position has been archived.

This known solution permits also the gradual reduction of the earlier set dose by turning the dose setting element made as a knob in the direction opposite to the dose setting direction. By using this dose resetting method, the total reset of the formerly set dose may be done by turning the knob manually with the same number of full turns as used when setting the dose.

From WO 2011025448 an injecting device is also known comprising a dose resetting mechanism, said device comprising a housing designed to be connected with a container comprising a medicine to be injected. This device comprises also a dose setting mechanism which is rotatably coupling with the housing in order to set a dose when it is turned in one direction, and to reset a dose when it is turned in the opposite direction. In order to reset a dose by turning a knob, another part of the resetting element should press a shaped projection located on a spring arm causing its disengagement from a projection in a driving unit. However, at the same time, during the reset the second part of the resetting element is moved because of the energy accumulated in the spring element, and the force acting onto the shaped projection located on the spring arm makes it once more engaged with the projection of the driving unit. Because of that, it is not possible to perform the automatic complete reset of the formerly set dose by a single turn of the dose setting mechanism by a determined angle. Moreover, the construction of this known solution extorts the application of limp and delicate resetting elements susceptible to deformations.

It is an object of this invention to construct an injecting device having a simply operated resetting mechanism which allows to automatically set an indicating element in its initial position by means of energy accumulated in a spring element, said energy being released by a dose setting element, without causing an axial motion of a threaded piston rod, so as to have the dose setting element motionless during a total automatic reset of a formerly set dose caused by a single limited motion of the dose setting element in the direction opposite to the dose setting direction.

An additional object of this invention is to eliminate all small and pliable elements taking part in both setting and resetting the dose, which are exposed to fractures, deformations or wearing down when the device is working, and thereby to decrease failure rates of the device and to increase the accuracy of dosing the administered medicine.

Another object of the invention is to diminish the time needed to carry out the complete reset, and simultaneously to simplify operating of the device during the reset.

Another object of the invention is to eliminate an out-of-control medicine outflow from the container during the full reset with a limited single movement of the dose setting element, by making a drive unit independent from the spring element after accumulating energy in it, so as not to allow for creating stresses in the drive unit, said stresses being transmitted onto the medicine container and causing an undesired dropping out of a medicine during this operation.

The device according to this invention is defined in the independent claim. Especially, the solution according to this invention is characterized in that the reset clutch is made as an axially slidable shaped coupling element, moved by means of a dose setting element, and moreover the reset clutch is functionally connected with the drive unit designed to co-operate with a spring means in such a way, that after releasing the reset clutch the drive unit is disconnected from the spring means with retained connection of this spring means with an indicating element, which is then automatically set in its stable starting position.

Preferably, the reset clutch connects in a detachable manner the ratchet with a coupling sleeve connected with the spring means as well as with the indicating element.

Advantageously, the reset clutch is axially slid by an axial movement of the clutch sleeve, said movement being related to the movement of the dose setting element in the direction opposite to the dose setting direction.

Advantageously, the reset clutch co-operates with the set of axial cams in such a way that the dose setting element moving in the direction opposite to the dose setting direction causes an axial travel of the clutch sleeve as well as the axial travel of the reset clutch.

Advantageously, the first axial cam is joined with the axially stabilized ratchet by a support surface of a bearing, and the second axial cam is joined with the axially slidable clutch sleeve.

Advantageously, the bearing is axially stabilized and secured against rotation in the housing by means of a ring projection and longitudinal projections.

Advantageously, a ratchet toothed wheel connected with a nut is located between the ratchet and the support surface of the bearing.

Advantageously, the nut and the ratchet are axially stabilized by opposite planes of the support surface of the bearing.

Advantageously, the ratchet has coupling projections to be slidably connected with the reset clutch.

Advantageously, the reset clutch is made as a splined ring.

Advantageously, the reset clutch is slidably set on the clutch sleeve in contact with the axial cam.

Advantageously, between the reset clutch and the coupling sleeve there is placed a reset spring pressing down this reset clutch to the ratchet and retaining it in mesh with the coupling projections.

Advantageously, the dose setting element is made as a knob rotatably coupling with the housing with a possibility to be rotated in the dose setting direction as well as in the opposite direction, enabling the rotary and sliding movement of the clutch sleeve.

Advantageously, the dose setting element is able to take any position in the operating range in relation to the indicating element having the fixed starting position.

Advantageously, the dose setting element makes less than one full turn in the direction opposite to the dose setting direction to ensure settlement of the indicating element in its starting position without causing the axial movement of the threaded piston rod. More advantageously, the dose setting element makes a rotary movement at the angle of less than 90°.

Advantageously, the spring means is made as a torsion spring.

Advantageously, the dose settling element made as a knob is protected against its axial movement in relation to the housing by means of a ring projection, and it is coupled with the clutch sleeve with the axial play enabling the axial sliding movement of said clutch sleeve in relation to the dose setting element when said dose setting element is moving in the direction opposite to the dose setting direction.

Advantageously, the axial play enabling the axial sliding motion of the clutch sleeve in relation to the dose setting element is smaller than the maximum axial travel caused by the relative turn of the axial cams and it is greater than the minimum travel of the reset clutch enabling the release of this reset clutch from engagement with the coupling projections.

By using a reset clutch in the form of a shaped coupling element axially slidable between the drive unit and the spring means of the shaped clutch element, a possibility is achieved to disconnect this drive unit from the spring means while preserving the connection of this spring means with the indicating element so that the released energy accumulated in said spring means causes automatically the back movement of the indicating element to its initial position without causing any axial movement of the threaded piston rod, that is without giving the set medicine dose.

The solution according to the invention enables simple and quick reset of the formerly set dose by releasing energy accumulated in the spring means and by making the indicating element to return to its zero initial position, said actions being initiated by one movement of the dose setting element in the direction opposite to that when setting a dose without the necessity of selectively correcting the dose by means of a series of operations gradually reducing the dose. Among others, it enables to quickly change a container with no loss of medicine. This quick and precise automatic reset is especially useful when one device is used for administration of different kinds of specific medicine, which often takes place for example in the insulin therapy, when selected containers with a medicine have to be changed often.

The complete and automatic reset carried out by the back motion of the dose setting element allows to carry out this operation in a quick and simple manner, especially by persons with reduced manual or visual efficiency, because there is no need to find and actuate an additional button when resetting the dose in the device according to this invention.

Enforcement of the axial movement of the reset clutch by means of a set of the axial cams makes one able to carry out the reset of the set dose by turning the dose setting element by the fixed value in the direction opposite to the dose setting direction, irrespective of the formerly set dose. After initiation by the user of the resetting motion by means of the dose setting element, it remains motionless. When the nut and the ratchet are axially set on the opposite planes of the support surface of the bearing, during resetting the force pressing this ratchet to the ratchet toothed wheel can be increased. The ratchet toothed wheel stabilized in that way stabilizes the nut, which diminishes piston rod vibration. Thanks to this additional stabilization of the drive unit, it is less susceptible to any disturbances from other parts of the mechanism which results in reduction of out-of-control outflow of the medicine from the container when the total reset is carried out.

An object of the invention is presented in its embodiment in the drawing in which

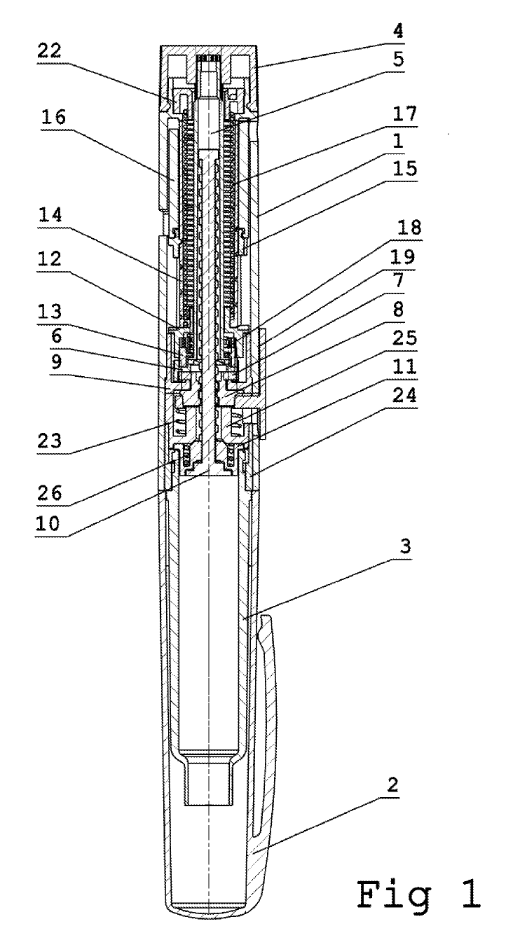

FIG. 1 presents an axial section of the injecting device;

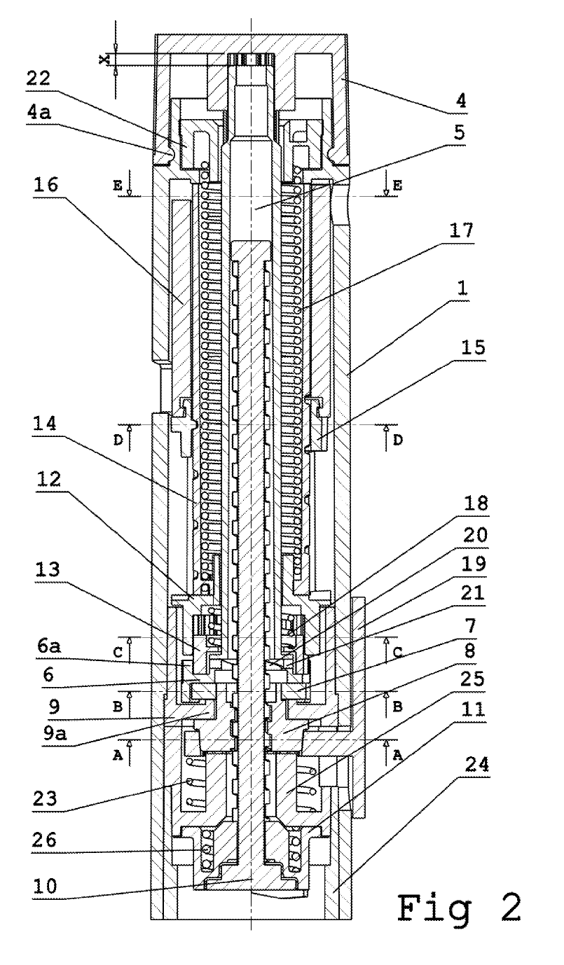

FIG. 2—an enlarged axial section of the device without a container housing and a cap, with a blocked threaded piston rod guide;

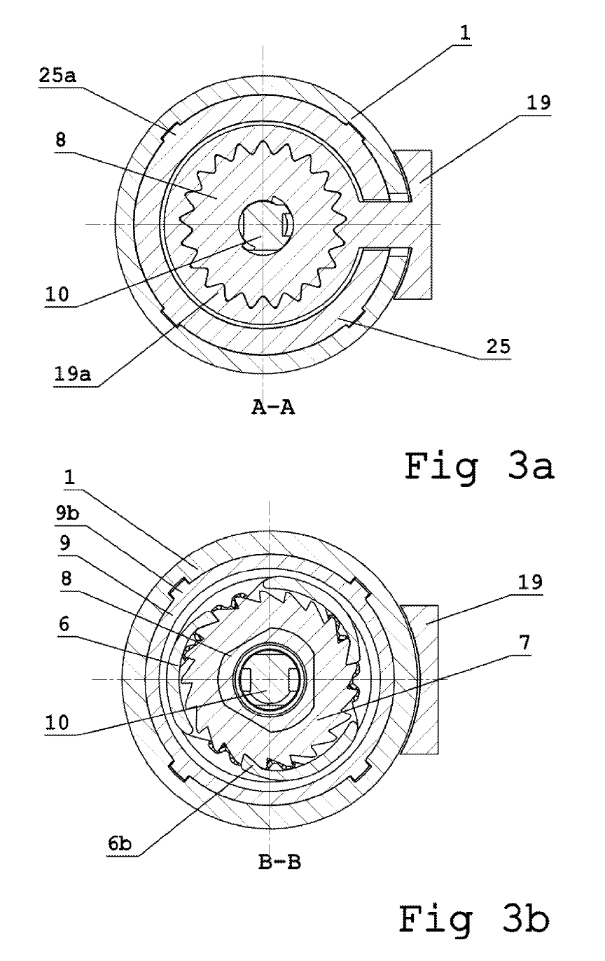

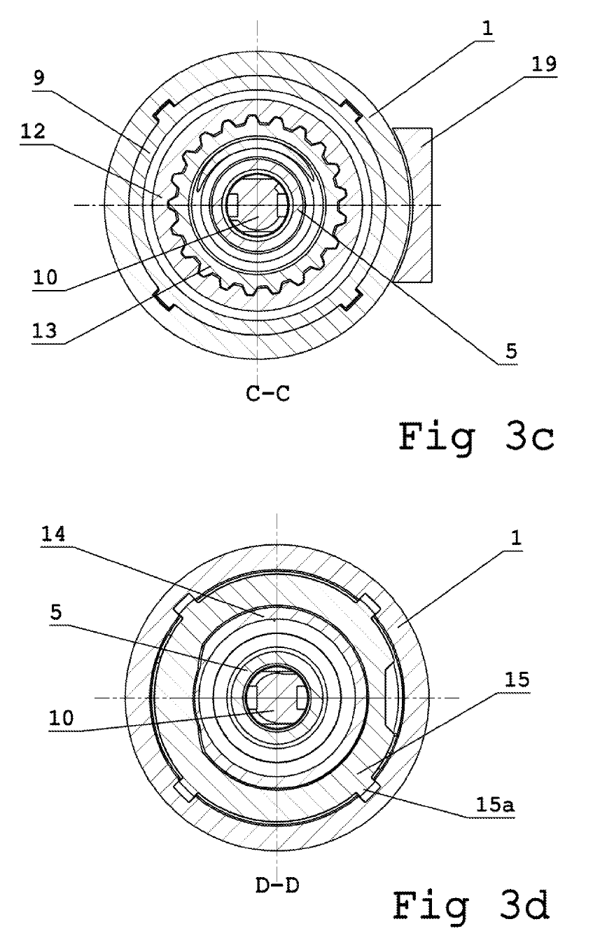

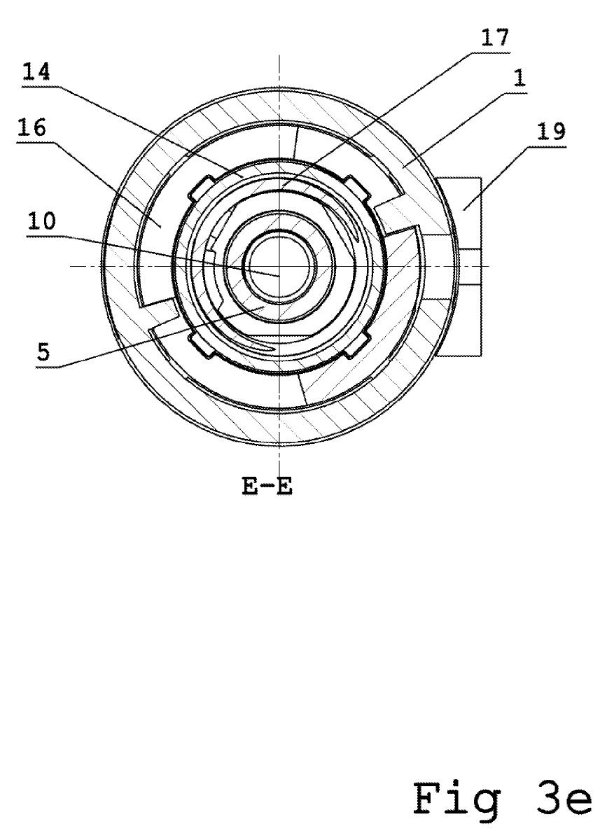

FIG. 3a—a cross-section through a nut along the line A-A marked in FIG. 2;

FIG. 3b—a cross-section through a ratchet toothed wheel along the line B-B marked in FIG. 2;

FIG. 3c—a cross-section through a reset clutch along the line C-C marked in FIG. 2;

FIG. 3d—a cross-section through a pulling nut along the line D-D marked in FIG. 2;

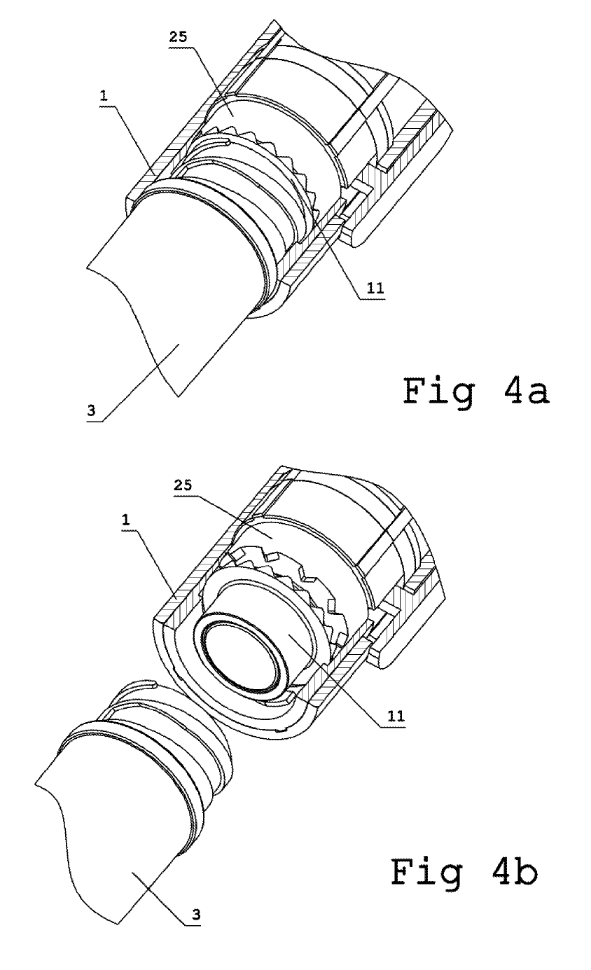

FIG. 3e—a cross-section through a part of an indicating element along the line E-E marked in FIG. 2; FIG. 4a—a partial longitudinal section through a housing showing a guide for a threaded piston rod together with an assembled housing of a container;

FIG. 4b—a partial longitudinal section through the housing showing a guide for the threaded piston rod with the housing of the container twisted out;

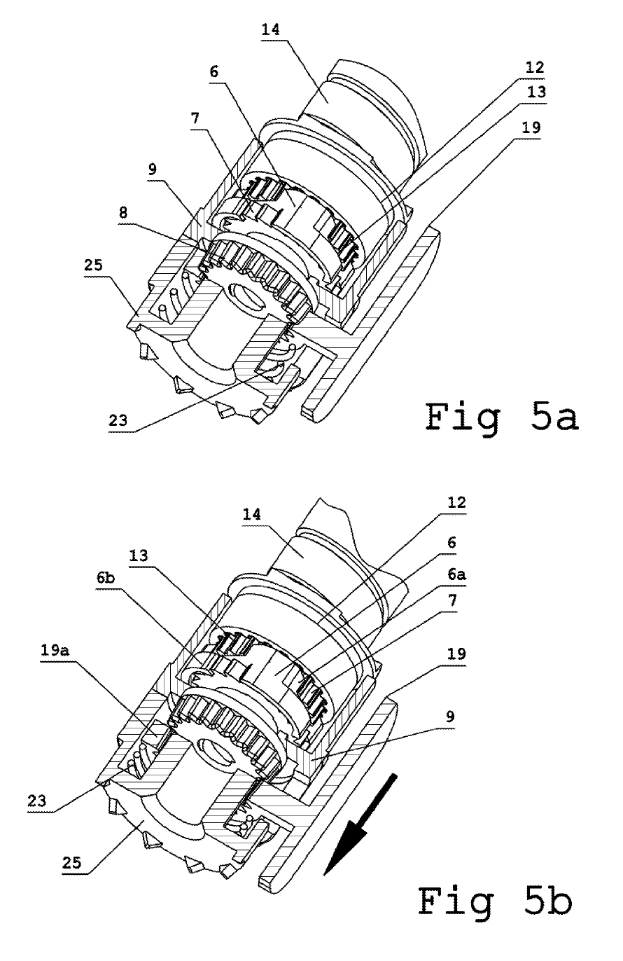

FIG. 5a—a partial longitudinal section through the housing showing the nut blocked by a trigger;

FIG. 5b—a partial longitudinal section through the housing showing the nut un-blocked by a trigger;

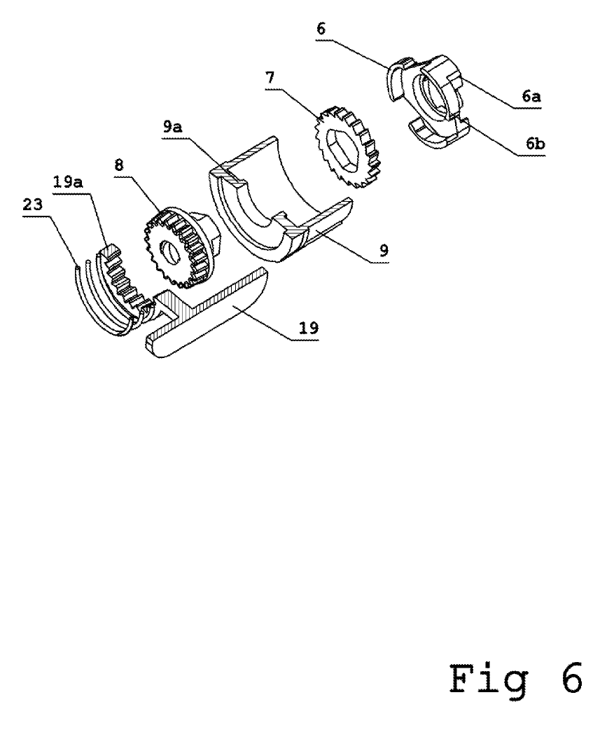

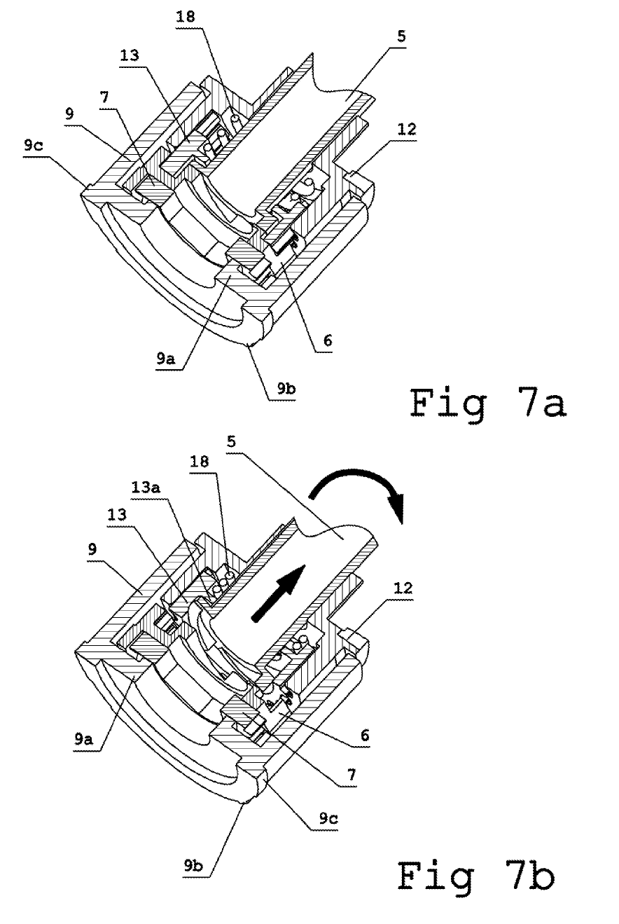

FIG. 6—an exploded perspective view of elements positioned axially and radially by a bearing; FIG. 7a—a detail with an axial section through a bearing with the connected reset clutch;

FIG. 7b—a detail with an axial section through a bearing with the reset clutch disconnected;

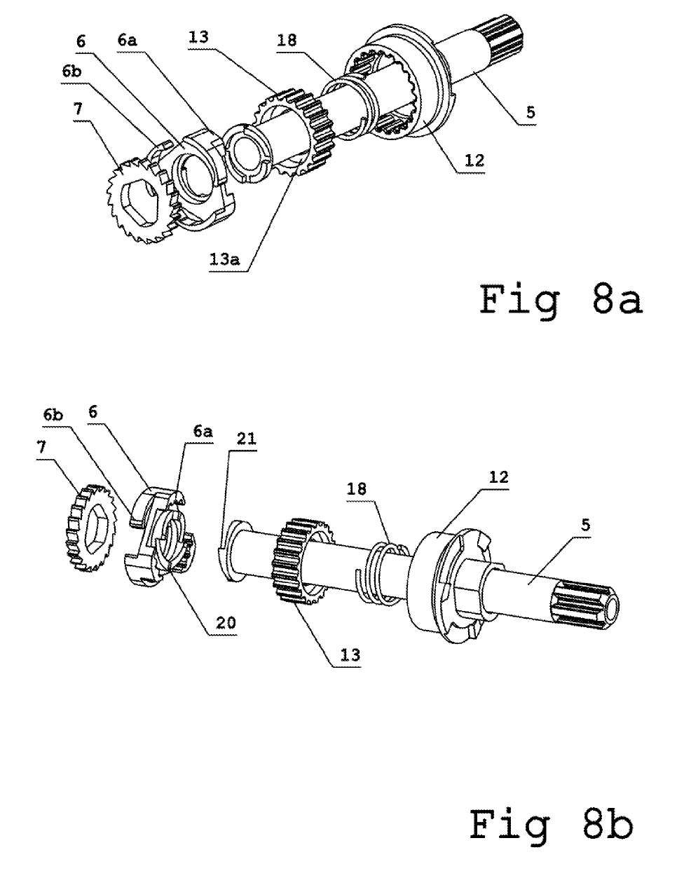

FIG. 8a—an exploded perspective view of elements co-operating with a reset clutch;

FIG. 8b—an exploded perspective view of elements co-operating with a reset clutch as in FIG. 8a, presented from another side;

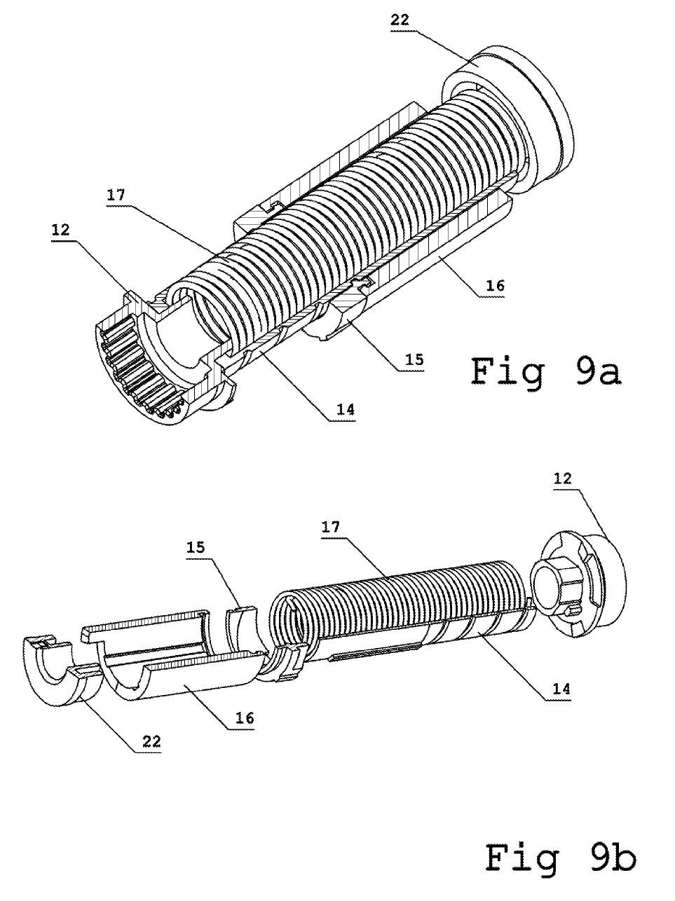

FIG. 9a—a partial longitudinal section of elements co-operating with a spring means and with an indicating element;

FIG. 9b—an exploded perspective view of the elements shown in FIG. 9a; and

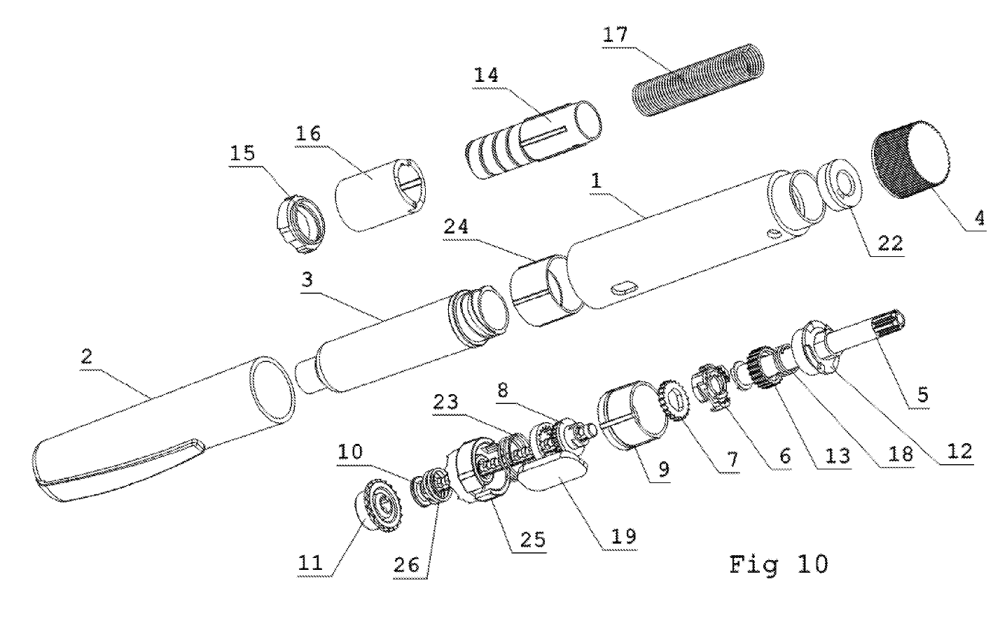

FIG. 10 presents an exploded perspective view of the whole device according to the invention.

As it is shown in FIG. 1, a housing 1 of a mechanism together with a cover cap 2 put on a housing of a container 3 looks as a writing device such as a pen. The housing of the container 3 protected by the cover cap 2 is intended to receive an exchangeable container with a medicine to be injected, not shown in the figure, and it is fastened at one side of the housing 1. A dose setting element 4 of an injected medicine is placed at the side opposite to the cover cap 2, said dose setting element being shaped as a knob rotatably coupling with the housing 1.

As it is shown in an enlarged view in FIG. 2, the dose setting element 4 is connected by a clutch sleeve 5 with a ratchet 6 co-operating with a ratchet toothed wheel 7, and during setting a dose it co-operates with consecutive cogs of this ratchet toothed wheel 7. The ratchet toothed wheel 7 is connected stiffly with a nut 8 rotatably coupled with a slide bearing 9 located motionlessly coaxially with an axis of the housing 1. A threaded piston rod 10 co-operating with the threaded nut 8 is slidingly located inside the clutch sleeve 5. The non-circular cross-section of the threaded piston rod 10 slidably co-operating with an opening made in a runner 11 allows for blocking of the rotary motion, and allows for longitudinal and axial movement of the threaded piston rod 10 inside the clutch sleeve 5. The ratchet toothed wheel 7 and the nut 8 are established in the axial direction by a support surface 9a of a slide bearing 9, said support surface separating said ratchet toothed wheel 7 and said nut 8, so that no axial forces are carried between the ratchet toothed wheel 7 and the nut 8, but only torques.

A drive unit comprising the ratchet 6, the nut 8 and the threaded piston rod 10 is separably engaged with a coupling sleeve 12 by means of an axially sliding reset clutch 13, and the coupling sleeve 12 is axially established inside the housing 1, and it is connected by a driving barrel 14 and a pulling nut 15 with a cylindrical indicating element 16, as well as with a spring means 17 made as a torsion spring. When the reset clutch 13 is axially shifted against the pressing force of a reset spring 18, no axial motion of the threaded piston rod 10 is caused because the nut 8 and the ratchet 6 are separated by the support surface 9a of the slide bearing, and moreover the rotary motion of the nut 8 is blocked by a trigger 19. When the dose setting element 4 is rotated clockwise, this spring means 17 is twisted in order to accumulate in it energy needed to inject the set medicine dose.

The clutch sleeve 5 is connected by a set of axial cams 20, 21 with the ratchet 6 having coupling projections 6a engaged with the reset clutch 13, and the outer teeth of the reset clutch 13 are engaged with inner teeth of the coupling sleeve 12. The reset clutch 13 is made advantageously as a splined ring having its cogs shaped on its outer surface, whereas the coupling projections 6a and the coupling sleeve 12 have appropriate shaped inner splines. The spring means 17, in its preferred solution of a torsion spring, has one of its ends connected by a blocking sleeve 22 to the housing 1, and its second end connected to the coupling sleeve 12. When rotating the dose setting element 4, made as a knob, clockwise, the spring means is twisted and energy needed to inject the set medicine dose or to carry on the automatic reset is gathered in said spring means 21.

The anticlockwise rotary motion of the dose setting element 4 in a limited angle range causes the angular displacement of the clutch sleeve 5 in relation to the ratchet 6 and causes a suitable rotation of the axial cams 20, 21, and in the result an axial motion of the clutch sleeve 5 covering a play x in relation to the dose setting element 4, wherein this axial motion is transferred onto the reset clutch 13 and makes the reset clutch 13 move into the coupling sleeve 12 until the reset clutch is totally disconnected from the coupling projections 6a of the ratchet 6. Only after such separation total automatic reset is carried out connected with the back movement of the cylindrical indicating element 16 to its initial position, forced by the energy accumulated in the spring means 17, wherein said automatic reset does not cause any rotation of the dose setting element 4. After the reset, the rotation of the dose setting element 4 by the same angle in the direction opposite to that when resetting causes a re-coupling of the reset clutch 13 and the ratchet 6, due to the pressure put by the reset spring 18, said ratchet 6 and the dose setting element 4 in this moment being located in relation to the indicating element 16 in the position depending on a set value before resetting. Thus, an initial position of the indicating element 16 does not depend on the constant and repeatable position of the dose setting position but it can adopt any values from the range in which the dose setting element 4 works.

The dose setting element 4 made as a knob is rotatably coupled at the end of the housing 1 and it is protected against axial shifting in relation to this housing by means of a ring projection 4a. An outer splines end part of the clutch sleeve 5 is located slidably in the inner splines of the dose setting element 4, with an axial play x. The axial play x limits the possibility of the axial displacement of the clutch sleeve 5, and in the result it is possible by a properly selected axial play x to limit the relative rotation of the axial cams 20, 21 to such a degree that they cannot achieve their maximum axial travel at which the axial cams can jump one over another. At the same time, this axial play x should be greater than the minimum travel of the reset clutch 13 that enables the release of this reset clutch 13 from the engagement with the coupling projections 6a.

The trigger 19 is located along the side surface of the housing 1 and it is able to block and unblock the nut 8. In a free connection, the trigger 19 is pressed by means of a spring 23 to the nut 8 and thanks to that, it blocks it against turning. The trigger is unblocked by sliding it axially against a pressing force exerted by the spring 23. Then the nut 8 is unblocked to release the energy formerly accumulated in the spring means 17 and to use this energy to slide the threaded piston rod 10, as well as to cause the complex movement of the cylindrical indicating element 16.

When the container housing 3 is fastened in a fastening sleeve 24, the runner 11 is pressed to a blockade 25 in order to protect it against rotating whereas, when unscrewing the container housing 3, the spring 26 acting in the axial direction causes uncoupling of the blockade 25 from the runner 11 which enables pressing the threaded piston rod 10 to its initial position and inserting a new medicine container. The threaded piston rod 10 may be pressed thanks to using on it, as well as on the nut 8, a not-self-locking thread.

In FIG. 3a there is shown a cross-section through the nut 8, the blockade 25 and the trigger 19 in the blocked position. As one can see in this figure, the blockade 25 has projections 25a protecting it against rotating in relation to the housing 1, and the trigger 19 has a blocking ring 19a having teeth inside it engaging with the teeth in the nut 8.

In FIG. 3b there is shown the cross-section through the ratchet toothed wheel 7 coupled with the nut 8. This ratchet toothed wheel 7 axially established in the bearing 9 is engaged on its circumference with three resilient arched arms 6b of the ratchet 6, whereas longitudinal projections 9b, protecting the bearing 9 against rotation, are fastened in suitable longitudinal grooves in the housing 1.

In FIG. 3c there is shown a cross-section of the reset clutch 13 being in engagement with the coupling sleeve 12. The reset clutch is made as a shaped clutch element having a form of a toothed wheel sliding on the clutch sleeve 5.

In FIG. 3d there is shown the pulling nut 15 having projections 15a located in the longitudinal grooves of the housing 1, said pulling nut 15 co-operating with an outer thread of the driving barrel 14.

In FIG. 3e there is shown a cross-section through a part of the cylindrical indicating element 16 engaged with the longitudinal projections of the driving barrel 14. In FIG. 1, FIG. 2, as well as in FIG. 3e the cylindrical indicating element is shown in its stable starting position.

When connecting the container housing 3 with the housing 1 of the mechanism, as it is shown in FIG. 4a, the teeth of the runner 11 are engaged with the grooves of the blockade 25, and the runner 11, protected in this manner against rotation, makes it able to block any rotational movement of the threaded piston rod 10. On the other hand, as it is shown in FIG. 4b, when disconnecting the container housing 3, the teeth of the runner 11 goes out of contact with the blockade grooves, and in effect of that the runner 11 can do its rotational motion when pressing the threaded piston rod 10 to the start position before inserting the new medicine container.

Before injecting the set medicine dose, the nut 8 is blocked by the trigger 19 pressed to it by means of the spring 23, as presented in FIG. 5a.

A stage in which a set dose is injected is shown in FIG. 5b, while in FIG. 6 there is shown the shape of the elements which are axially and radially set by the bearing 9. By the axial sliding motion of the trigger 19 and by overcoming a pressing force of the spring 23, the nut 8 is released from its engagement with the inner toothed blocking ring 19a. When the trigger 19 is in this position, the nut 8 rotated by the energy accumulated in the spring means 17 pushes the threaded piston rod 10 out, and in effect of this, a medicine dose set before is pushed out from the replaceable medicine container not shown in the drawing.

While the set medicine dose is being injected, after a slide of the trigger 19, the energy accumulated in the spring means 17 is transferred into the nut 8 successively by the coupling sleeve 12, the reset clutch 13, the coupling projections 6a, the resilient angle arms 6b, and the ratchet toothed wheel 7. The energy accumulated in the spring means 17, when released, makes also the cylindrical indicating element 16 to move in a complex manner, and this element after injecting the set medicine dose returns to its initial position marked by an unambiguous zero indication on the cylindrical indicating element 16. Both during setting a determined medicine dose, and when injecting it, the axially sliding reset clutch 13 connects by the pressing force of the reset spring 18 the coupling sleeve 12 with the ratchet 6, as it is shown in FIG. 7a.

In FIG. 7b there is shown an operation of releasing the reset clutch 13. One should turn the clutch sleeve 5 by an angle of about 90 degrees in the direction opposite to that when setting the dose. This turning direction is marked by an arc arrow. The clutch sleeve 5 has a multiprojection end part on one of its sides, said end part being designed to be coupled with a knob forming the dose setting element 4, and on its other side it has the axial cam 21, which is in contact with the axial cam 20 shaped on the ratchet 6. During rotating these axial cams one in relation to another, when the clutch sleeve 5 is rotated in the direction opposite to the dose setting direction, these axial cams 20, 21 repulse themselves mutually in the axial direction, causing the sliding movement of the reset clutch 13, up to disconnecting it from the ratchet 6. The sliding direction of the clutch sleeve 5, the axial cam 21 and the reset clutch 13 is marked by an arrow located in the axis of the clutch sleeve 5. In effect of the movement of the reset clutch 13, the reset spring 18 located on the clutch sleeve 5 is pressed between the coupling sleeve 12 and the inner ring 13a of the reset clutch 13.

The axial cams 20, 21, shown in the embodiment in the drawing, are made as four-segmented ones, so the maximum lift during their rotation in relation to each other is obtained at an angle of 90 degrees. In such a case, the injecting device is, for the user's convenience, equipped with any limiter making the axial cams 20, 21 not able to move in relation to each other by a greater angular value. The function of a such limiter is fulfilled for example by the suitable selection of the axial play x between the clutch sleeve 5 and the dose setting element 4 shown in FIG. 2. When using the axial cams 20, 21 shaped as one-segment parts, which are not shown in the drawings, the rotation of the dose setting element 4 in the direction opposite to that used when setting the dose should be limited to an angle smaller than 360 degrees.

The slide bearing 9 has the longitudinal projections 9b and a ring projection 9c by means of which this bearing is fastened in the housing 1. However, the stabilizing action of the ratchet toothed wheel 7, being pressed to the support surface 9a of the slide bearing 9, is caused by the reset spring 18. When the reset spring 18 is compressed by moving the axial cams 20, 21 during the resetting operation, the force pressing the ratchet toothed wheel 7 to the support surface 9a is increased, which additionally protects against transferring vibrations created during this operation onto the nut 8, as well as onto the piston rod.

The shape of the elements shown in FIG. 7a and FIG. 7b without the slide bearing 9 is depicted in FIG. 8a and FIG. 8b in the exploded perspective view in two different angular positions. The reset clutch 13, as it is shown in FIG. 8a and FIG. 8b, is shaped in its advantageous embodiment as a splined wheel having the inner ring 13a intended to the frontal contact with the axial cam 21 from one side and with the reset spring 18 from the other side.

As it is shown in FIG. 9a and FIG. 9b, the coupling sleeve 12 is connected with the driving barrel 14, as well as with one end of the spring means 17, shaped as a torsion spring, connected with its second end to the blocking sleeve 22 designed to be fastened in the housing 1. When the coupling sleeve 12 is turned during setting the dose, the driving barrel 14 is rotating and the spring means 17 is twisted causing energy accumulation in it. The pulling nut 15 co-operates with the thread on the driving barrel 14, said pulling nut 15 being set by the longitudinal projections in longitudinal grooves inside the housing 1. This pulling nut 15 slides during setting a dose along the housing 1 of the mechanism, and it pulls with itself the cylindrical indicating element 16 which turning together with the driving barrel 14 and sliding together with the pulling nut 15 moves along a screw line in relation to the housing 1. However, when resetting the set dose, the pulling nut 15 co-operating with the driving barrel 14 pushes off the cylindrical indicating element 16 to its starting position.

The shapes of all parts of the injecting device according to the invention are better illustrated in FIG. 10.

The device according to the invention enables, when resetting the set dose, the automatic return of the cylindrical indicating element 16 to its zero position, that means to its stable initial position, by means of energy accumulated in the spring means 17 without any sliding motion of the trigger 19 and without starting the sliding motion of the axial threaded piston rod 10, and in the result without injecting the set medicine dose. This effect has been achieved because the release of the reset clutch 13 permits to release the turning motion of the coupling sleeve 12 connected with the driving barrel 14 driving the cylindrical indicating element 16 as well as with the spring means 17.

The mechanism resetting a set dose in the injecting device according to the invention is started by turning the dose setting element 4 in the direction opposite to the dose setting direction. Turning the dose setting element 4 by an angle of approximately 90 degrees in the anticlockwise direction forces the clutch sleeve 5 to slide in the direction to the dose setting element 4, causing the axial movement of the shaped reset clutch 13, said movement causing the uncoupling of the ratchet 6 from the coupling sleeve 12. When turning the dose setting element 4, one is causing that the axial cams 20, 21 are mutually pushed back, and that the pressing force of the reset spring 18 is overcome. In the result of such disconnecting, the threaded piston rod 10 is not moved because that motion is blocked by the trigger 19, however the cylindrical indicating element 16 driven by the driving barrel 14 and by the pulling nut 15 is moved to its initial position, and the spring means 17 is released to its initial preload.

In all figures of the drawing a torsion spring is shown as the spring means 17, however the invention is not limited to that form of the spring element. In the solution according to the invention all kinds of means accumulating energy by deformation may be used, in particular spiral springs, helical springs, as well as elastomer springs.

1. An injecting device comprising a dose resetting mechanism, said device comprising:
a housing as well as a container housing for receiving a container with a medicine to be injected;
a threaded piston rod mounted inside the housing and co-operating with a runner enabling axial shift of the threaded piston rod;
a drive unit comprising a ratchet, a nut, as well as the threaded piston rod axially stabilized in the housing and having a thread fitted to the thread of the nut;
a dose setting element functionally connected to the housing, and connected by means of a clutch sleeve with the ratchet functionally connected with the nut and co-operating with an indicating element, which in its initial position has a fixed starting position;
a spring means connected with the housing as well as with the dose setting element in such a way, that an action of the dose setting element causes the energy accumulation in said spring means;
a reset clutch;
a trigger displaceable in relation to the housing and designed to release energy accumulated in the spring means by releasing the nut and the threaded piston rod characterized in that the reset clutch is made as an axially sliding, shaped coupling element moved by means of the dose setting element, and moreover this reset clutch is functionally connected with the drive unit destined to co-operate with the spring means in such a way that when this reset clutch is released, said drive unit is disconnected from the spring means retaining the connection of this spring means with the indicating element which automatically takes then the fixed starting position.

2. A device according to claim 1, characterized in that the reset clutch in a detachable manner connects the ratchet with a coupling sleeve being connected with the spring means and with the indicating element.

3. A device according to claim 1, characterized in that the reset clutch is axially shifted by the axial movement of the clutch sleeve resulting from the movement of the dose setting element in the direction opposite to the dose setting direction.

4. A device according to claim 1, characterized in that the reset clutch co-operates with the set of axial cams in such a way that the movement of the dose setting element in the direction opposite to the dose setting direction causes an axial travel of the clutch sleeve as well as the axial travel of the reset clutch.

5. A device according to claim 4, characterized in that the first axial cam is joined with the ratchet stabilized axially by a support surface of a bearing, and the second axial cam is joined with the axially slidable clutch sleeve.

6. A device according to claim 5, characterized in that the bearing is immobilized axially and protected against rotation in the housing by means of a ring projection and longitudinal projections.

7. A device according to claim 5, characterized in that between the ratchet and the support surface there is located a ratchet toothed wheel connected with the nut.

8. A device according to claim 1, characterized in that the nut and the ratchet are axially stabilized by the opposite planes of the support surface of a bearing.

9. A device according to claim 1, characterized in that the ratchet has coupling projections to be slidably connected with the reset clutch.

10. A device according to claim 1, characterized in that the reset clutch is shaped as a splined ring.

11. A device according to claim 1, characterized in that the reset clutch is slidably located on the clutch sleeve in contact with an axial cam.

12. A device according to claim 1, characterized in that between the reset clutch and a coupling sleeve a reset spring is located, the spring pressing this reset clutch to the ratchet and holding it engaged with coupling projections.

13. A device according to claim 1, characterized in that the dose setting element is shaped as a knob which is rotatably coupled on the housing with the possibility to be rotated in the dose setting direction as well as in the opposite direction, in order to enable a rotary and an axial sliding movement of the clutch sleeve.

14. A device according to claim 13, characterized in that the dose setting element is designed to take any position in the operating range in relation to the indicating element 40 being in its stable starting position.

15. A device according to claim 1, characterized in that the dose setting element makes less than one full rotation in the direction opposite to the dose setting direction, said rotation setting the indicating element in the stable starting position without causing any axial motion of the threaded piston rod.

16. A device according to claim 15, characterized in that the dose setting element makes a rotary motion in the angle range of less than 90 degrees.

17. A device according to claim 1, characterized in that the spring means is shaped as a torsion spring.

18. A device according to claim 1, characterized in that the dose setting element shaped as a knob is protected against the axial sliding motion in relation to the housing by means of a ring projection, and it is engaged with the clutch sleeve with an axial play (x) left in order to enable an axial sliding motion of this clutch sleeve in relation to the dose setting element when the dose setting element is moved in the direction opposite to the dose setting direction.

19. A device according to claim 18, characterized in that the axial play (x) enabling the axial sliding motion of the clutch sleeve in relation to the dose setting element is smaller than the maximum axial travel caused by the relative rotation of axial cams.

20. A device according to claim 18, characterized in that the axial play (x) enabling the axial sliding movement of the clutch sleeve in relation to the dose setting element is greater than the minimum axial travel of the reset clutch allowing for release of the reset clutch from its engagement with the coupling projections.
